(19) United States
(12) Patent Application Publication
(10) Pub. No.: US 2020/0051690 A1
(43) Pub. Date: Feb. 13, 2020

(54) METHODS AND SYSTEMS TO FACILITATE USER THERAPY ADHERENCE VIA VIRTUAL ENVIRONMENT GROUP INTERACTIONS BASED ON SIMILAR THERAPEUTIC DEVICE USAGE

(71) Applicant: KONINKLIJKE PHILIPS N.V., Eindhoven (NL)

(72) Inventors: NATHANIEL SOLOMON FOX, PITTSBURGH, PA (US); MANUEL LAURA LAPOINT, PITTSBURGH, PA (US); LEONARDO ALBERTO BALOA WELZIEN, LAKE FOREST, PA (US)

(21) Appl. No.: 16/658,982

(22) Filed: Oct. 21, 2019

Related U.S. Application Data
(63) Continuation of application No. 14/006,337, filed on Sep. 20, 2013, now abandoned.
(60) Provisional application No. 61/466,490, filed on Mar. 23, 2011.

(51) Int. Cl. G16H 40/63; G16H 80/00; G06Q 10/10

(52) U.S. Cl. CPC G16H 40/63; G06Q 10/10; G16H 80/00

(57) ABSTRACT

In certain embodiments, user therapy adherence may be facilitated via virtual environment group interactions that are based on similar therapeutic device usage. In some embodiments, a virtual world may be provided to enable users to interact with one another in the virtual world. Usage information associated with the users may be obtained from physical therapeutic devices (e.g., in an autonomous and/or periodic manner), where the usage information associated with a given user includes information indicating how a respective physical therapeutic device is used by the given user. A virtual world group for the virtual world may be generated based on the usage information associated with the users. In some embodiments, a physical therapeutics device of at least one of the group users may present an indication of the placement in the virtual world group to facilitate interaction of the group users in the virtual world.

### RELATED APPLICATION(S)

This application is a continuation of U.S. patent application Ser. No. 14/006,337, filed Sep. 20, 2013, which is a national stage application of International Application No. PCT/IB12/50918, filed Feb. 28, 2012, which claims priority to U.S. Provisional Application No. 61/466,490, filed Mar. 23, 2011, each of which is incorporated herein by reference in its entirety.

### FIELD OF THE DISCLOSURE

The present disclosure pertains to methods and systems to increase subject adherence to a therapy regimen.

### BACKGROUND

Virtual environments configured to facilitate interaction between subjects of different types of therapies exist. For example, one or more virtual environments configured to facilitate interaction between subjects of respiratory therapies involving an airway pressure device are known.

### SUMMARY

In certain embodiments, user therapy adherence may be facilitated via virtual environment group interactions that are based on similar therapeutic device usage. In some embodiments, a virtual environment may be provided to enable users to interact with one another in the virtual environment. Usage information associated with the users may be obtained from physical therapeutic devices (e.g., in an autonomous and/or periodic manner). As an example, the usage information associated with a given user may include information indicating how a respective physical therapeutic device is used by the given user. A group for the virtual environment may be generated based on the usage information associated with the users. In some embodiments, a physical therapeutics device of at least one of the group users may present an indication of the placement in the group to facilitate interaction of the group users in the virtual world.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of any limits.

### BRIEF DESCRIPTION OF THE DRAWINGS

FIG. 1 schematically illustrates a system to promote user adherence to a therapy regimen, in accordance with one or more embodiments; and

FIG. 2 illustrates a method for promoting user adherence to a therapy regimen, in accordance with one or more embodiments.

### DETAILED DESCRIPTION

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

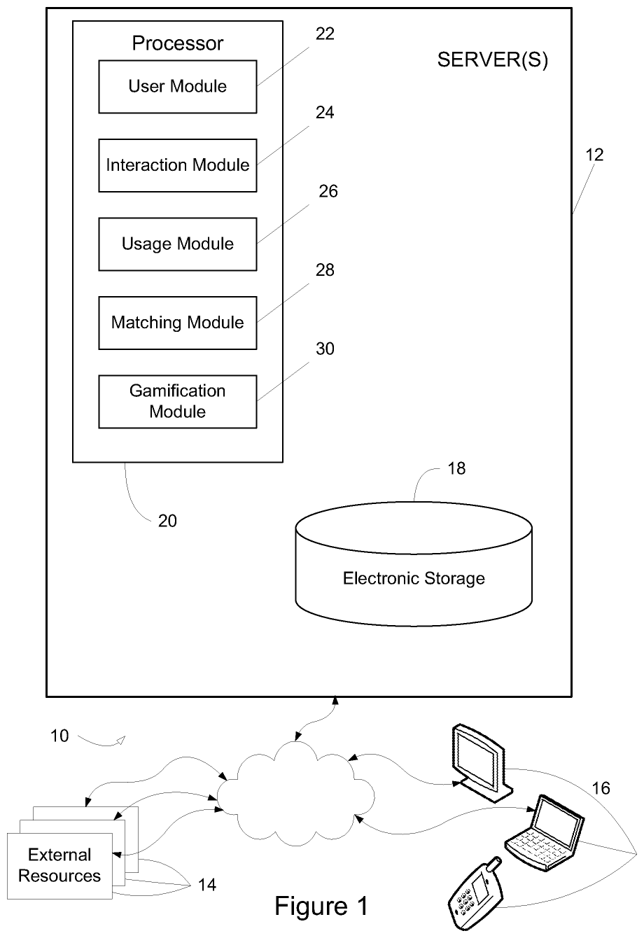

FIG. 1 schematically illustrates a system 10 configured to promote user compliance with a therapy regimen. Adherence to a therapy regimen involving a (positive) airway pressure device (a.k.a. PAP device) may be challenging for many subjects. System 10 is configured to provide a virtual environment to subjects that facilitates, and encourages interactions between subjects of a common therapy or therapy type. These interaction may be used by subjects to encourage each other, exchange helpful tips or hints that make compliance easier, more efficient, and/or less obtrusive, answer questions about therapy, maintain accountability to peers, and/or otherwise promote compliance. The virtual environment may be dedicated to facilitating interactions between therapy subjects, or the virtual environment may be a more general purpose environment with one or more dedicated areas, groups, walls, forum, and/or other content segments dedicated to interactions between therapy subjects.

As used herein, a “virtual environment” may refer to an environment provided to users over a network. The virtual environment may enable synchronous and/or asynchronous interactions, public, private, and/or semi-private interactions, text-based interactions, audio-based interactions, video-based interactions, and/or other interactions. By way of non-limiting example, a virtual environment may include one or more of a virtual world or space, an network/online forum, a chat room, a (private) instant messaging service, an SMS messaging service, a social networking web site, a micro-blogging service, submission-based user and/or multi-user messaging, a user and/or multi-user wall, status postings, group-sourced answers to posted questions, a blog hosting service, and/or other virtual environments.

System 10 may include one or more servers 12, and/or other components. System 10 may operate in communication and/or coordination with one or more external sources 14. Users may interface with system 10 and/or external resources 14 via client computing platforms 16. The components of system 10, servers 12, external resources 14, and/or client computing platforms 16 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which servers 12, external resources 14, and/or client computing platforms 16 may be operatively linked via some other communication media.

A given client computing platform 16 may include one or more processors configured to execute computer program modules. The computer program modules may be configured to enable one or more users associated with the given client computing platform 16 to interface with system 10 and/or external resources 14, and/or provide other functionality attributed herein to client computing platforms 16. By way of non-limiting example, the given client computing platform 16 may include one or more of a desktop computer, a laptop computer, a handheld computer, a NetBook, a Smartphone, a gaming console, and/or other computing platforms. Alternatively, and/or simultaneously, a given client computing platform 16 may be integrated in or embedded in a user's therapeutic device, such as a PAP device.

External resources 14 may include sources of information, hosts and/or providers of virtual environments outside of system 10, external entities participating with system 10, therapeutic devices, and/or other resources. In some implementations, some or all of the functionality attributed herein to external resources 14 may be provided by resources included in system 10. For example, a user's therapeutic device may interface with system 10 via a client computing platform 16.

The server 12 may be configured to provide, or cooperate with client computing platforms 16, to provide a virtual environment to users. This may include hosting, serving, and/or otherwise providing a virtual space to the users within which (anonymous) interaction between users is facilitated via client computing platforms 16. The server 12 may include electronic storage 18, one or more processors 20, and/or other components. The server 12 may include communication lines, or ports to enable the exchange of information with a network and/or other computing platforms.

Electronic storage 18 may comprise electronic storage media that electronically stores information. The electronic storage media of electronic storage 18 may include one or both of system storage that is provided integrally (i.e., substantially non-removable) with server 12 and/or removable storage that is removably connectable to server 12 via, for example, a port (e.g., a USB port, a FireWire port, etc.) or a drive (e.g., a disk drive, etc.). Electronic storage 18 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 18 may store software algorithms, information determined by processor 20, information obtained, identified, gathered, and/or provided by one or more computer program modules, information received from server 12, information received from client computing platforms 16, information received from external resources 14, and/or other information that enables server 12 to function properly.

Processor(s) 20 is configured to provide information processing capabilities in server 12. As such, processor 20 may include one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some implementations, processor 20 may include a plurality of processing units. These processing units may be physically located within the same device, or processor 20 may represent processing functionality of a plurality of devices operating in coordination.

As is shown in FIG. 1, processor 20 may be configured to execute one or more computer program modules. The one or more computer program modules may include one or more of a user module 22, an interaction module 24, a usage module 26, a matching module 28, a gamification module 30, and/or other modules. Processor 20 may be configured to execute modules 22, 24, 26, 28, and/or 30 by software; hardware; firmware; some combination of software, hardware, and/or firmware; and/or other mechanisms for configuring processing capabilities on processor 20.

It should be appreciated that although modules 22, 24, 26, 28, and 30 are illustrated in FIG. 1 as being co-located within a single processing unit, in implementations in which processor 20 includes multiple processing units, one or more of modules 22, 24, 26, 28, and/or 30 may be located remotely from the other modules. The description of the functionality provided by the different modules 22, 24, 26, 28, and/or 30 described below is for illustrative purposes, and is not intended to be limiting, as any of modules 22, 24, 26, 28, and/or 30 may provide more or less functionality than is described. For example, one or more of modules 22, 24, 26, 28, and/or 30 may be eliminated, and some or all of its functionality may be provided by other ones of modules 22, 24, 26, 28, and/or 30. As another example, processor 20 may be configured to execute one or more additional modules that may perform some or all of the functionality attributed below to one of modules 22, 24, 26, 28, and/or 30.

It will be appreciated that the illustration of modules 22, 24, 26, 28, and/or 30 being executed solely on processor 20 separate from client computing platforms 16 and external resources 14 is not intended to be limiting. For example, in some implementations, the client computing platforms 16 may be configured to provide locally at least some of the functionality attributed above to one or more of modules 22, 24, 26, 28, and/or 30. Similarly, one or more of modules 22, 24, 26, 28, and/or 30 may be executed locally on individual client computing platforms 16 while others are executed on server 12.

In certain embodiments, user module 22 is configured to manage personal user information. Personal user information may include stated information, information received from a therapeutic device, information entered by a caregiver of the user, information derived by analysis, and/or other information. For example, personal user information may include user identification, user gender, user age, user age range, user geographical information, user demographic information, user ethnicity and/or race, and/or other personal user information. Personal user information may also include account information, which may include stated information, stated (communication/interaction) preferences, information received from a therapeutic device, information entered by a caregiver of the user, information derived by analysis, and/or other information. For example, account information may include a username and a pass code (or password), which may be used to authenticate access to system 10. While user information (and account information) may identify a user in a non-virtual environment (e.g. the real world), the operation of the preferred embodiment of system 10 is intended to preserve anonymity among users in the virtual environment.

Interaction module 24 is configured to provide or host a virtual environment to users such that the users are able to interact with each other in the virtual environment. The virtual environment may include one or more of an online forum, a chat room, private instant messaging, submission-based user and/or multi-user messaging, a user and/or multi-user wall, status postings, group-sourced answers to posted questions, and/or other virtual environments, for example implemented through online communications. The virtual environment may include achievement rankings based on information gathered regarding interactions through a virtual environment and/or based on information gathered from a user's usage of a therapeutic device. Interaction through a virtual environment may foster a sense of community among users. For example, users may collaborate by voting for (or ‘liking’) the best advice from a set of available answers to a posted question. Interaction module 24 may be configured to track or monitor a user's interactions in the virtual environment over a period of time in order to derive user preferences. For example, a user preference may include a particular way over communicating and/or interaction with other users, a preferred timeslot to engage in the virtual environment, and/or other user preferences. Alternatively, and/or simultaneously, user preferences may be explicitly stated by a user. In certain embodiments, the interactions in the virtual environment are implemented in such a way as to maintain a user's anonymity.

Usage module 26 is configured to obtain usage information associated with users, wherein the usage information associated with a given user represents usage of a therapeutic device by the given user in accordance with a therapy regimen. Usage module 26 may obtain usage information by receiving transmission from one or both of a therapeutic device (which may be an airway pressure device such as CPAP, BiPAP, etc.) and/or a client computing platform. Usage information may include one or more of a therapy starting date, user experience level, an average duration of daily usage, a usage pattern, metrics indicating quality of therapy and/or quality of sleep, level of compliance with a therapy regimen, therapeutic device characteristics (e.g. mask type or PAP device settings), derived usage characteristics, user-stated usage characteristics, and/or other usage information. User-stated usage characteristics may include issues or problems the user is experiencing while undergoing therapy, and/or other user-stated usage characteristics. Derived usage characteristics may include low and/or irregular usage (as detected e.g. through analysis of usage reports), decreasing usage, excessive mask leak, and/or other derived usage characteristics. In certain implementations, a PAP device used by a user may (autonomously and/or periodically) compile a usage report and submit it to server 12. For example, through recorded usage reports (which may be representative of such information as a user's sleep habits) the usage module may be further configured to assign and/or update a therapy rank (e.g. representative of compliance with a therapy regimen) and/or an experience rank to a user (e.g. based on the therapy starting date and/or the accumulated usage of a therapeutic device). Therapy rank and/or experience rank may be used by the computer program modules of processor 20.

Matching module 28 is configured to identify a match between a group of two or more users based on the usage information associated with the users in the group, and to communicate the match to the users in the group to facilitate interaction in the virtual environment between users within the group. In certain embodiments, identification of a match is further based on (personal) user information, (derived or stated) user preferences, and/or other information, from any of the computer program modules of processor 20. In certain embodiments, the operation of the matching module may further be based on a user's past experience within a virtual environment, or a user's (pattern of) past usage of a therapeutic device. For example, users may be matched based on having a similar (level of) experience with a particular therapy regimen, therapeutic device, and/or usage characteristics (such as issues or problems experienced while undergoing therapy). Alternatively, and/or simultaneously, users may be matched based on having a different (level of) experience. In the latter case, a more experienced user could act as a mentor to a novice user, and thus promote adherence through assisting the novice user. Having a mentor who has overcome issues or problems similar to those a novice user is currently facing, may be particularly helpful to a novice user. Such a similarity may be detected by comparing patterns of usage information between users. Operation of matching module 28 may further be based on a user's therapy rank and/or experience rank.

Once matching module 28 has identified a match, the match may be communicated to the users in a group through an instant massage, a text message, an email message, a status posting, and/or other types of communication suitable to prompt a user to take action (and thus suitable to facilitate interaction in a virtual environment). A suitable type of communication may be based on the type of virtual environment used and/or a user's communication preferences. For example, matching module 28 may communicate a match by sending an email message, optionally including an active hyperlink to a website hosting a virtual environment. As another example, in a case where client computing platform 16 is integrated or embedded in a PAP device, matching module 28 may communicate a match by displaying a pop-up window on an interface of the PAP device, with an invitation to interact in a virtual environment.

Gamification module 30 is configured to gather interaction information for a user regarding interactions within the virtual environment, and provide incentives to the user based on the gathered interaction information. The term “gamification” is used to indicate the process of encouraging certain user behaviors through the bestowal of status and/or value within the virtual environment and/or the real world. This may have the effect of turning activities that are not necessarily related to a game into a game. In certain embodiments, the operation of the matching module may be further based on the gathered interaction information. For example, within a virtual environment, a point system may be implemented. Points may be rewarded for gained experience, achievements, interaction between users, activity within the virtual environment, and/or other desirable behavior conceivably tied to improved user adherence to a therapy regime. Achievement rankings may be included in or shared through the virtual environment. Points may be redeemed within the virtual environment, e.g. in exchange for different visual themes, an avatar, virtual items and/or abilities for an avatar, moderation powers over certain user interactions, and/or other virtual goods within the virtual environment. Operation of gamification module 30 may further be based on a user's therapy rank and/or experience rank.

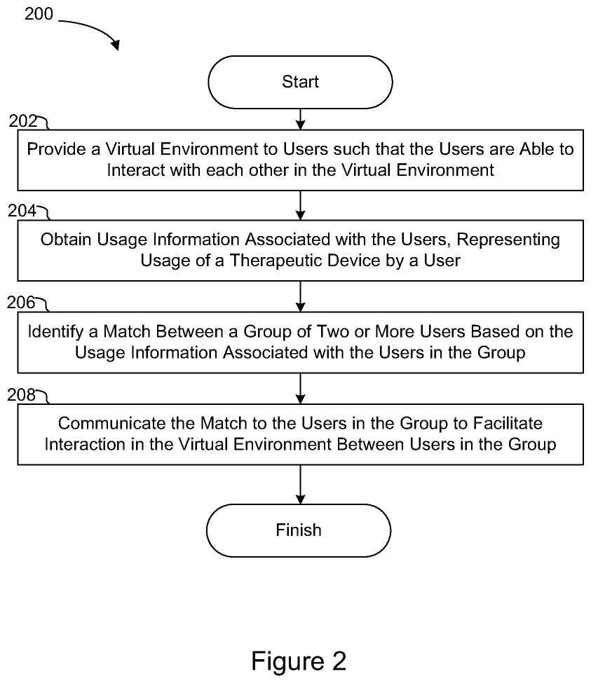

FIG. 2 illustrates a method 200 of promoting user adherence to one or more therapy regimen. The operations of method 200 presented below are intended to be illustrative. In some embodiments, method 200 may be accomplished with one or more additional operations not described, and/or without one or more of the operations discussed. Additionally, the order in which the operations of method 200 are illustrated in FIG. 2 and described below is not intended to be limiting.

In some embodiments, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

At an operation 202, a virtual environment may be provided to users such that users are able to interact with each other in the virtual environment. This may include presenting views of the environment that can be presented to the users. In some implementations, operation 202 may be performed by an interaction module similar to or substantially the same as interaction module 24 (shown in FIG. 1 and described above).

At an operation 204, usage information associated with the users may be obtained, representing usage of a therapeutic device by a user. In some implementations, operation 204 may be performed by a usage module similar to or substantially the same as usage module 26 (shown in FIG. 1 and described above).

At an operation 206, a match between a group of two or more users may be identified, based on the usage information associated with the users in the group. In some implementations, operation 206 may be performed by a matching module similar to or substantially the same as matching module 28 (shown in FIG. 1 and described above).

At an operation 208, the match may be communicated to the users in the group to facilitate interaction in the virtual environment between users in the group. The type of communication used may depend on the type of virtual environment used. For example, in certain embodiments the match may be communicated by sending an email to the users in the group. In some implementations, operation 208 may be performed by a matching module similar to or substantially the same as matching module 28 (shown in FIG. 1 and described above).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the embodiments have been described in detail for the purpose of illustration based on what is currently considered to be most practical and preferred, it is to be understood that such detail is solely for that purpose and that the disclosure is not limited to these embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A system for facilitating user therapy adherence via virtual world group interactions that are based on similar therapeutic device usage, the system comprising:
a computer system comprising one or more processors programmed with computer program instructions that, when executed, cause the computer system to:
provide a virtual world configured to represent users as avatars in the virtual world and to enable the users to interact with one another in the virtual world;
autonomously and periodically obtain, from physical therapeutic devices, usage information associated with the users, wherein the usage information associated with a given user includes information indicating how a respective physical therapeutic device is used by the given user;
generating a virtual world group for the virtual world based on the usage information associated with the users such that a first user and a second user of the users are placed to the virtual world group based on the usage information associated with the first user and the usage information associated with the second user having similar therapeutic device usage information; and
cause a physical therapeutics device of the first user or the second user to present an indication of the placement in the virtual world group to facilitate interaction of the first user and the second user in the virtual world.

2. The system of claim 1, wherein the physical therapeutics device comprises a positive airway pressure device of the first user or the second user.

3. The system of claim 1, wherein the physical therapeutic devices monitor one or more physiological parameters of the users.

4. The system of claim 1, wherein the computer system is caused to provide the indication of the placement in the virtual world group to the physical therapeutics device to cause the physical therapeutic device to present the indication of the placement in the virtual world group.

5. The system of claim 1, wherein the usage information comprises one or more of an average duration of daily usage, a usage pattern, or derived usage characteristics.

6. The system of claim 1, wherein the virtual world is configured to enable one or more users to communicate with one another via one or more of a chat room, private instant messaging, submission-based user and/or multi-user messaging, a user and/or multi-user wall, or status postings.

7. A method implemented by one or more processors executing computer program instructions that, when executed, perform the method, the method comprising:
providing a virtual environment configured to represent users as avatars in the virtual environment and to enable the users to interact with one another in the virtual environment;
periodically obtaining, from physical therapeutic devices, usage information associated with the users, wherein the usage information associated with a given user includes information indicating how a respective physical therapeutic device is used by the given user;
placing a first user and a second user of the users into a group for the virtual environment based on the usage information associated with the first user and the usage information associated with the second user having similar therapeutic device usage information; and
causing a device of the first user or the second user to present an indication of the placement in the group to facilitate interaction of the first user and the second user in the virtual environment.

8. The method of claim 7, wherein periodically obtaining the usage information associated with the users comprises autonomously and periodically obtaining the usage information associated with the users from the physical therapeutic devices.

9. The method of claim 7, wherein the device comprises a physical therapeutic device of the first user or the second user.

10. The method of claim 7, wherein the device comprises a positive airway pressure device of the first user or the second user.

11. The method of claim 7, wherein the physical therapeutic devices monitor one or more physiological parameters of the users.

12. The method of claim 7, wherein causing the physical therapeutic device to present the indication of the placement in the group comprises providing the indication of the placement in the group to the physical therapeutics device.

13. The method of claim 7, wherein the usage information comprises one or more of an average duration of daily usage, a usage pattern, derived usage characteristics, or user-stated usage characteristics.

14. A non-transitory computer-readable media comprising instructions that, when executed, cause operations comprising:
providing a virtual environment configured to enable users to interact with one another in the virtual environment;
obtaining, from physical therapeutic devices, usage information associated with the users, wherein the usage information associated with a given user includes information indicating how a respective physical therapeutic device is used by the given user;
placing a first user and a second user of the users into a group for the virtual environment based on the usage information associated with the first user and the usage information associated with the second user having similar therapeutic device usage information; and
causing a physical therapeutic device of the first user or the second user to present an indication of the placement in the group to facilitate interaction of the first user and the second user in the virtual environment.

15. The media of claim 14, wherein obtaining the usage information associated with the users comprises periodically obtaining the usage information associated with the users from the physical therapeutic devices.

16. The media of claim 14, wherein obtaining the usage information associated with the users comprises autonomously and periodically obtaining the usage information associated with the users from the physical therapeutic devices.

17. The media of claim 14, wherein the physical therapeutic device comprises a positive airway pressure device of the first user or the second user.

18. The media of claim 14, wherein the physical therapeutic devices monitor one or more physiological parameters of the users.

19. The media of claim 14, wherein causing the physical therapeutic device to present the indication of the placement in the group comprises providing the indication of the placement in the group to the physical therapeutics device.

20. The media of claim 14, wherein the usage information comprises one or more of an average duration of daily usage, a usage pattern, or derived usage characteristics.
